(19) United States
(12) Patent Application Publication
(10) Pub. No.: US 2014/0107439 A1
(43) Pub. Date: Apr. 17, 2014

(54) BIOLOGICAL OPTICAL MEASUREMENT DEVICE, STIMULUS PRESENTATION METHOD, AND STIMULUS PRESENTATION PROGRAM

(71) Applicants: Hirokazu Atsumori, Tokyo (JP); Hiroki Sato, Tokyo (JP); Masashi Kiguchi, Tokyo (JP)

(72) Inventors: Hirokazu Atsumori, Tokyo (JP); Hiroki Sato, Tokyo (JP); Masashi Kiguchi, Tokyo (JP)

(73) Assignee: HITACHI, LTD., Tokyo (JP)

(21) Appl. No.: 14/117,154

(22) PCT Filed: Jun. 15, 2012
(86) PCT No.: PCT/JP2012/065323

§ 371 Date: Dec. 20, 2013

(30) Foreign Application Priority Data
Jun. 17, 2011 (JP) 2011-134727

(51) Int. Cl. A61B 5/16; A61B 5/1455

(52) U.S. Cl. CPC A61B 5/165; A61B 5/14551
USPC 600/322; 600/558

(57) ABSTRACT

Disclosed is a device using a biological optical measurement technology to evaluate mood states in daily life of an examinee by presenting a first task once or a plurality of times and then presents a second task a plurality of times, calculating a hemoglobin signal of a predetermined measurement point for the first task and a hemoglobin signal of a predetermined measurement point for the second task, and calculating quantitative values using the obtained hemoglobin signals.

### BACKGROUND

The present invention relates to a device for supporting evaluation of the mood state of an examinee based on the measurement data of a biological optical measurement device.

In recent years, there has been an approach to find out the features of the mental state including individual mood and feelings, from biological measurement results. A typical research using functional magnetic resonance imaging (fMRI) is disclosed in Non-patent document 1 (Gray, et al., “Integration of emotion and cognition in the lateral prefrontal cortex,” Proc. Natl. Acad. Sci. U.S.A, 99(6), 4115-4120 (2002)). The research is conducted by presenting videos to induce the emotional states of healthy subjects, and measuring the prefrontal cortex activity of the subjects who carry out a task called “n-back task”, to show the results of the evaluation of the mood states. The n-back task is a task requiring a function of human working memory (WM). In this research, the prefrontal cortex activity associated with a verbal n-back task and a non-verbal n-back task has been studied to find out features that are influenced by both pleasant and unpleasant emotional states. However, in the measurement of fMRI, the examinee to be measured is detained and exposed to extremely loud noise, so that the environment is uncommon in daily life. Thus, the measurement environment of fMRI may influence the mental state including mood and feelings of the examinee, differently from the everyday situations. Further, for example, Patent document 1 (Japanese Unexamined Patent Application Publication No. Hei9 (1997)-98972) describes a biological optical measurement device for measuring the inside of a living body by using light with a plurality of wavelengths in the range from visible to infrared, and transforming the information of the inside of the living body into a two-dimensional image. The biological optical measurement device described in this document is designed to generate light by semiconductor laser diodes, irradiate a plurality of parts of the examinee by guiding the generated light thought optical fiber bundles, detect the light transmitted or reflected from the inside of the body of the examinee, guide the detected light to photodiodes through the optical fibers, and transform the biological information, such as blood circulation, hemodynamic changes, and hemoglobin concentration changes, into a two-dimensional image. Such a biological optical measurement device has a feature that it is non-invasive and less restrictive to the living body. Thus, the biological optical measurement device is suitable for evaluating the mental state and biological information of an individual under the conditions of daily life, compared to large-scale brain activity measurement technologies such as fMRI.

For example, Patent document 2 (Japanese Unexamined Patent Application Publication No. 2009-285000) describes a method for evaluating the mental state and biological information under the conditions of daily life by using such a biological optical measurement technology. Similar to the fMRI research described above, the method described in this document is designed to give a verbal WM task (requiring the phonological loop) and a non-verbal WM task (not requiring the phonological loop) to use the human working memory (WM) function, and measure the prefrontal cortex activity by the biological optical measurement technology. In this method, the task different from the n-back task is used. In other words, the n-back task is designed to allow the examinee to memorize and retain stimuli displayed on a screen in series while answering by recalling the stimulus presented several items before. However, in the verbal and non-verbal WM tasks used in this document, the memorization and retention is separated from the recall in terms of time in the execution of the WM tasks.

The feature of the prefrontal cortex activity described in this document is that the prefrontal cortex activity associated with the memorization and retention of the verbal WM task, and the score of “depression-dejection” (POMS_D) obtained from a short version of the standardized questionnaire POMS (Profile of Mood State) show a negative correlation, in which there is a poor correlation with the recall. On the other hand, there is no significant correlation with POMS_D in the prefrontal cortex activity associated with the non-verbal WM task. However, the both WM tasks basically reflect similar cognitive processes including memorization and retention. Thus, it is proposed to show the relative value calculated from the prefrontal cortex activity associated with the verbal and non-verbal WM tasks, as the quantitative value that can be compared between examinees.

The advantages of the method of this document are as follows. One is that there is no need to induce changes in mood state before measurement. The other is that non-restrictive and non-invasive biological optical measurement technology is used. Thus, it is expected that a self-check system for the mental health care can be developed by objectively obtaining an biomarker reflecting daily mood states.

### SUMMARY

The biological optical measurement technology for making the brain activity state being visible is expected to be applied to providing information on the individual's mental states such as mood and feelings. The biological optical measurement technology can be used under the conditions of daily life, compared to the large-scale brain functional imaging technology such as fMRI. Conventionally, there has been proposed a method for evaluating daily mood states by obtaining biological signals with respect to a plurality of types of cognitive tasks by using the biological optical measurement technology. However, a method for presenting cognitive tasks and a series of measurement protocols have not been established.

A biological optical measurement device according to the present invention includes: one or a plurality of light irradiation parts for irradiating an examinee with light; one or a plurality of light detection parts for detecting light transmitted or reflected from the examinee; a plurality of measurement points formed by a plurality of combinations of the light emitting part and the light detection part; a stimulus presentation part for presenting a plurality of different types of tasks to the examinee; a calculation part for calculating hemoglobin signals based on the changes in the concentrations of oxygenated hemoglobin and deoxygenated hemoglobin inside the examinee, from the intensity of the light detected by the light detection part; a storage part for storing the hemoglobin signals; and various tables showing the task type, the presentation order, and the like, in the storage part. The stimulus presentation part presents a first task once or a plurality of times, and then presents a second task a plurality of times. Then, the calculation part calculates hemoglobin signals of predetermined measurement points for the first task, as well as hemoglobin signals of predetermined measurement points for the second task, to calculate quantitative values by using the obtained hemoglobin signals.

By using a biological optical measurement device according to the present invention, it is possible to evaluate mood states under the conditions of daily life. Further, it is also possible to optimize protocols to obtain the mood states.

### BRIEF DESCRIPTION OF THE DRAWINGS

FIG. 1 is a block diagram of a biological optical measurement device according to an embodiment of the present invention;

FIG. 2 is a graph stored in a storage part of the biological optical measurement device according to an embodiment of the present invention;

FIG. 3 is a graph stored in the storage part of the biological optical measurement device according to an embodiment of the present invention;

FIGS. 4A and 4B are views of an example of the probe of the biological optical measurement device according to an embodiment of the present invention;

FIG. 5 is a view of an example of a presentation sequence of a spatial working memory task;

FIG. 6 is a view of an example of a presentation sequence of a verbal working memory task;

FIGS. 7A and 7B are views of a presentation order of the spatial and verbal working memory tasks;

FIG. 8 is a view of an example of oxygenated Hb and deoxygeneraterd Hb signals;

FIGS. 9A, 9B, and 9C are examples of correlation maps between brain activities in the execution of the working memory tasks and mood questionnaire scores;

FIG. 10 shows equations described in an embodiment;

FIG. 11 is a table for storing mood indices;

FIGS. 12A and 12B are examples of a mood index display;

FIGS. 13A and 13B are examples of a mood index display;

FIG. 14 is an example of a mood index display;

FIG. 15 is an example of a mood index display;

FIGS. 16A, 16B, and 16C are examples of a mood index display;

FIGS. 17A and 17B are examples of tables in which mood indices and marks are associated with each other;

FIG. 18 is an example of a measurement setting screen;

FIG. 19 is a flow chart illustrating the procedure of an embodiment;

FIG. 20 is an example of a present sequence of working memory tasks; and

FIG. 21 is an example of a presentation sequence of the working memory tasks.

### DETAILED DESCRIPTION

In the present invention, there is proposed a device for optimizing a biological optical measurement technology to evaluate daily mood states. More specifically, it is based on the following knowledge obtained by the inventors.
(1) It is defined the order of presenting a non-verbal WM task a plurality of times and then presenting a verbal WM task plurality of times, as an order A. Further, it is defined the order of presenting the verbal WM task a plurality of times and then presenting the non-verbal WM task a plurality of times, as an order B. Then, it is found that the quantitative value of the prefrontal cortex activity associated with the memorization and retention of the verbal WM task in the order A, is significantly higher than that in the order B in terms of negative correlation with depressed mood.
(2) There is no statistical difference found in the quantitative value of the prefrontal cortex activity for the memorization and retention of the non-verbal WM task between the orders A and B.

With the knowledge described above, the inventors clarified that the order A is suitable for obtaining the quantitative value of the prefrontal cortex activity reflecting depressed mood. Based on this finding, a specific embodiment of a biological optical measurement device disclosed by the present invention will be described in detail below with reference to the accompanying drawings. The present embodiment performs a mood evaluation under normal environment by using biological optical measurement, which may not be achieved by a large-scale brain function measurement device such as fMRI.

In brief, the mood evaluation uses the knowledge that the brain activity signal reflecting the memorization and retention of the verbal working memory (WM), reflects the daily mood of healthy individuals, which is described in the “supporting research” below.

### Supporting Research

The inventors obtained knowledge to solve the problem from the results of the study described below that conducted the biological optical measurement and the mood score assessment with questionnaire for 40 healthy individuals.

### <Method>

### <Biological Optical Measurement>

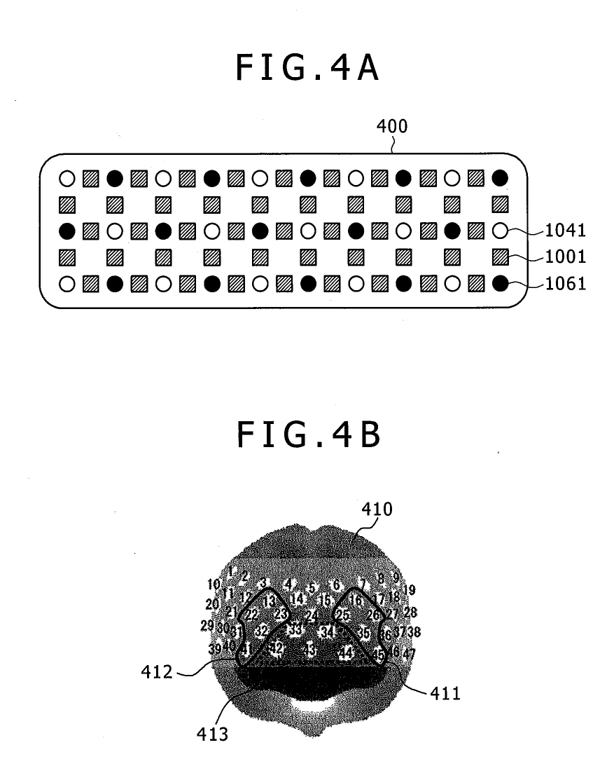

FIG. 4A shows a 3×10 biological optical measurement probe 400 in which 15 light irradiation points 1041 and 15 light detection points 1061 are alternately arranged. The biological optical measurement probe 400 is set in the prefrontal regions to obtain hemoglobin (Hb) signals as brain activity data from 47 measurement channels (ch). At this time, the position of each measurement point 1001 in a cerebral cortex surface 410 is as shown in FIG. 4B, in which the channel numbers 1 to 47 are given to the respective measurement points 1001. In particular, the regions corresponding to the left and right dorsolateral prefrontal cortex (DLPFC) are surrounded by solid lines 411 and 412, and the region corresponding to the frontal pole around the central part of the prefrontal area is surrounded by a dotted line 413. Two types of tasks, a spatial working memory (WM) task and a verbal WM task, are presented to the examinee to evaluate the brain activity with respect to each of the tasks.

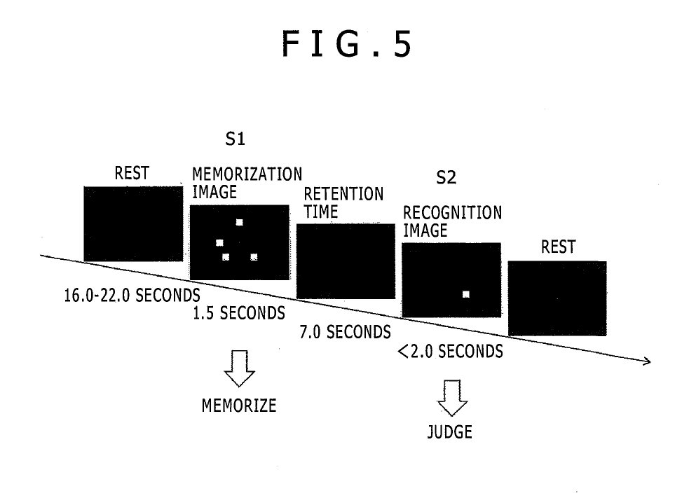

The outline of the spatial WM task is shown in FIG. 5. A memorization image (S1) includes squares placed at 8 locations around the central fixation point, in which 4 or 2 locations are white squares and others are gray squares. The memorization image (S1) is presented for 1.5 seconds. Here, these expressions are referred to as 4-itme condition and 2-item condition, respectively, according to the number of white squares. FIG. 5 is an example of the 4-item condition. After 7 seconds passed, a recognition image (S2) in which only one of the 8 locations is the while square is presented. The examinee is instructed to retain the positions of the white squares of the first memorization image S1. The examinee judges whether the white square of the recognition image S2 matches with any of the white squares memorized in the first image. Note that the number of tasks is 8 both for the 4-item condition and for the 2-item condition. Further, the tasks are presented in random order.

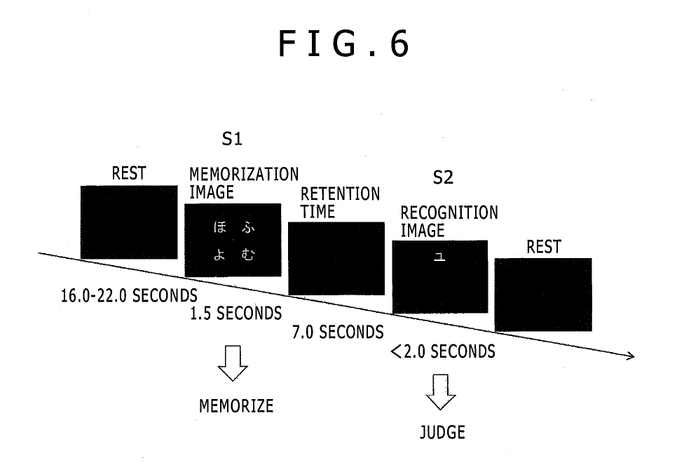

The outline of the verbal WM task is shown in FIG. 6. A memorization image (S1) includes hiragana characters written at 4 or 2 locations around the central fixation point. The memorization image (S1) is presented for 1.5 seconds. Here, these expressions are referred to as 4-item condition and 2-item condition according to the number of hiragana characters. FIG. 6 is an example of the 4-item condition. After 7 seconds passed, a recognition image (S2) in which one katakana character is displayed is presented. The examinee memorizes the characters of the first memorization image S1, and judges whether the katakana of the next presented recognition image S2 matches with any of the characters memorized in the first image. Different types of characters in S1 and S2 are used to make the examinee judge the memory based on the phonological features instead of the morphological information of the characters. Note that the number of tasks is 8 both for the 4-item condition and for the 2-item condition. Further, the tasks are presented in random order.

The examinees answer both the spatial and verbal WM tasks by a controller or a mouse button.

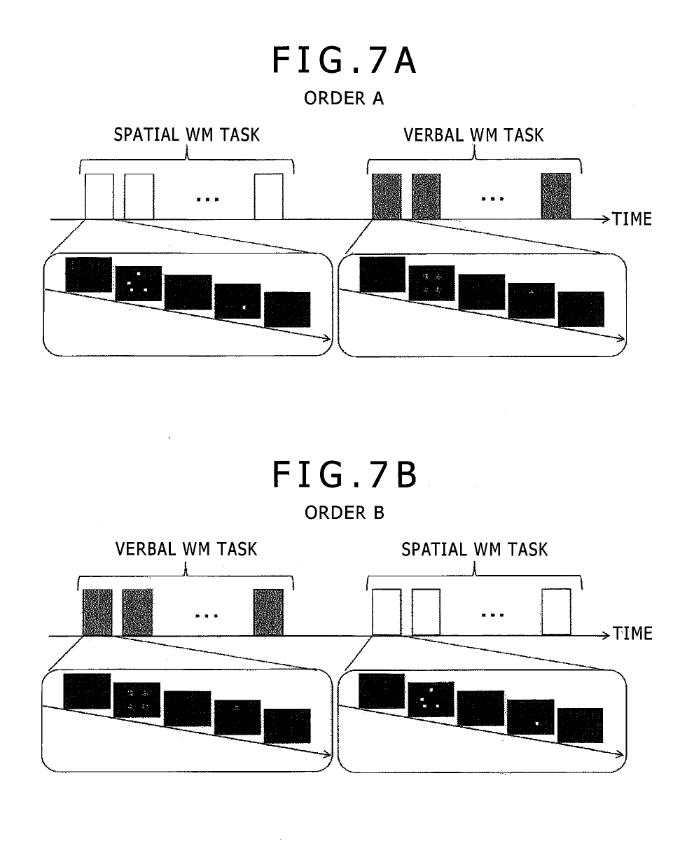

Further, the order in which each WM task is carried out is counterbalanced by the examinees. In other words, half of the examinees should first carry out the spatial WM tasks, and next the verbal WM tasks after the completion of the first tasks in the order A shown in FIG. 7A. The remaining half of the examinees should first carry out the verbal WM tasks, and next the spatial WM task after the completion of the first tasks in the order B shown in FIG. 7B.

In the analysis, oxygenated and deoxygenated Hb signals are obtained from the time-series data measured in each ch for each examinee. A time of 8.5 seconds from the presentation of the first image (S1) of the WM task to the presentation of the second image (S2) is defined as the task time. Then, a time of 25.5 seconds, in which 1 second before the task time and 16 seconds after the task time are added to the task time, is taken out as one block. The data of each block is baseline-corrected by a first order line fitted for the data of the first 1 second and the last 4 seconds in each block. It goes without saying that the time to be taken out as one block is not limited to the above example, and the length of the task time as well as the acquisition time before and after the task can be changed. Further, the time after 5 to 8.5 seconds from the presentation of S1 is defined as the activity time. Then, the mean value of the oxygenated Hb signal in the activity time is obtained in each block. Further, a t value is calculated from the mean value of all the blocks, which is defined as the “brain activity value”.

### <Questionnaire>

In order to evaluate the relationship between the brain activity state and the mood of the examinee, the POMS scores reflecting the mood states for the past one week was obtained by using the standardized questionnaire “POMS short version” (“Guide to a short version of the POMS and representative case descriptions” written by Kazuhito Yokoyama, Tokyo: Kaneko Publishing Inc., 2005.) to evaluate the mood of the examinees. The questionnaire allows the examinees to select one from 5 different levels: “not at all”, “a little”, “moderately”, “quite a bit”, and “extremely”, corresponding to their mood for each of the 30 items such as “tense”, “vigorous”, and “sad”. From the answers, the POMS scores of the mood of the examinees were obtained in 6 criteria: “tension-anxiety”, “depression-dejection”, anger-hostility”, “vigor”, “fatigue”, and “confusion”.

### <Result>

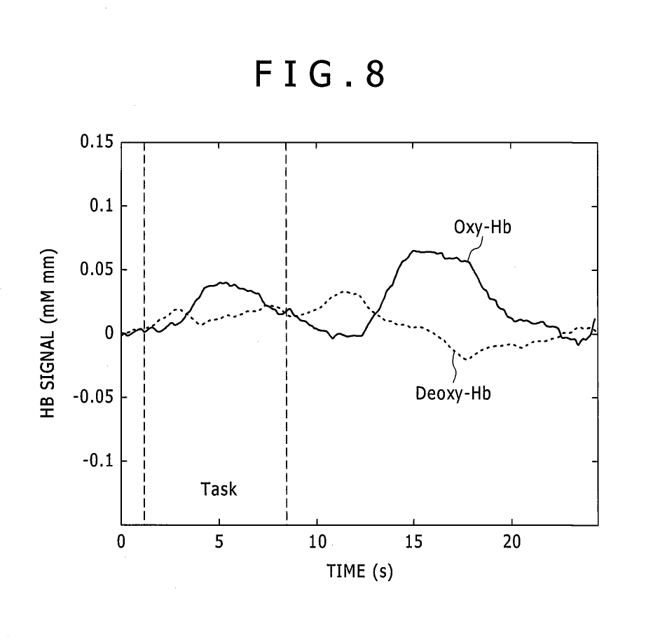

As a result of the study of the Hb singles, the task-related increase in the oxygenated Hb signal to the spatial and verbal WM tasks, and the task-related decrease in the deoxygenated Hb signal to the spatial and verbal WM tasks are observed (FIG. 8). Main activity regions are the regions corresponding to the left and right dorsolateral prefrontal cortex (DLPFC). It is known that DLPFC, which is the region including the middle frontal gyrus (Brodmann area 46, or BA46) and the like, is activated by the WM tasks. The spatial characteristics of the brain activity are similar in the two task conditions and the difference in those characteristics between the spatial WM task and the verbal WM task was not observed. Further, the difference in the time-series data of Hb signals in the activity regions between the tasks was not observed.

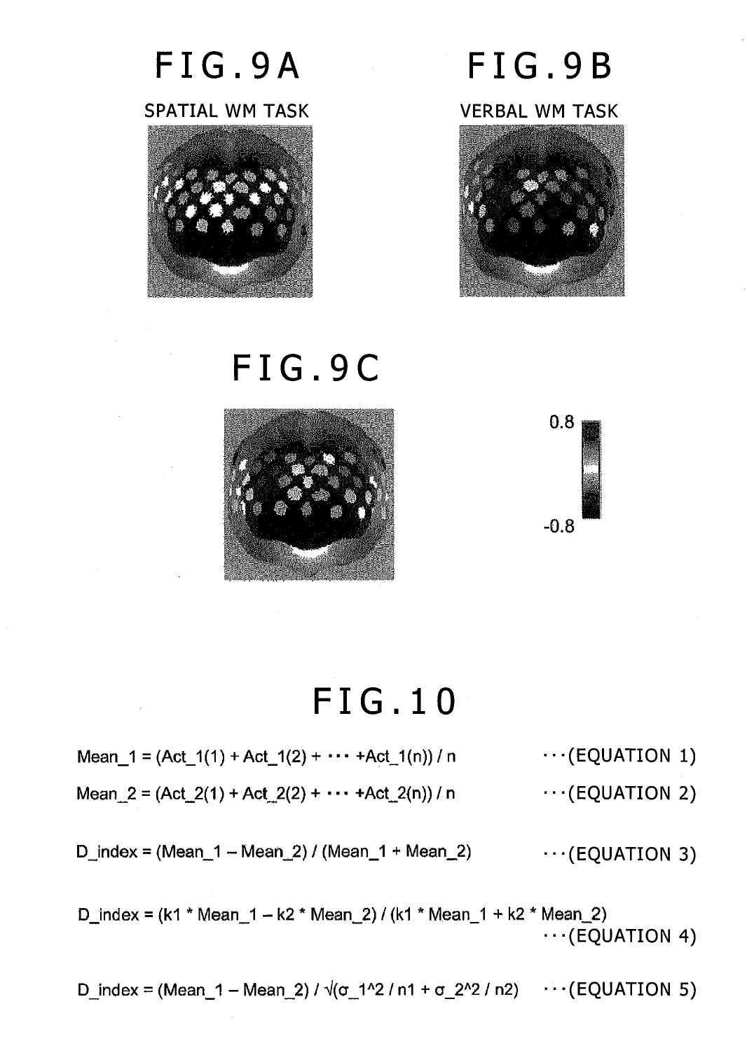

Next, the correlation between the brain activity values (t values) and the POMS scores was analyzed in each order of the tasks (the orders A and B, see FIGS. 7A and 7B). The results show the statistically significant negative correlation coefficients between the brain activity values of the verbal WM task and POMS depression score in the order A (first: spatial WM task, later: verbal WM task), but the weak and statistically insignificant positive correlation coefficients between the brain activity values of the spatial WM task and the POSM depression score (FIGS. 9A, 9B). On the other hand, the difference between the tasks, as that observed in the order A, was not observed in the order B (first: verbal WM task, later: spatial WM task).

Further, based on the results of the order A described above, t values indicating the difference of brain activities between the tasks using the mean value of each block of the spatial WM task and that of the verbal WM task was calculated. As a result, those t values show a statistically significant positive correlation with the POMS depression score at the measurement points corresponding to DLPFC 411 and 412 (FIG. 9C).

The results show that when the correlation between the brain activities associated with the spatial/verbal WM task and the POSM depression score is evaluated, the brain activity values associated with the spatial WM task does not have statistically significant correlations regardless of the order, while the brain activity values associated with the verbal WM task have statistically significant correlations in the order A rather than in the order B. As described above, this is a new method for obtaining more accurate mood states by optimizing the task presentation order when the brain activity values are obtained for the two types of WM tasks. The inventors clarified that the index related to depression can be obtained by optimizing the presentation order of different tasks.

Based on the knowledge described above, specific configuration and procedure of a biological optical measurement device to achieve the above goals will be described as embodiments of the present invention.

### First Embodiment

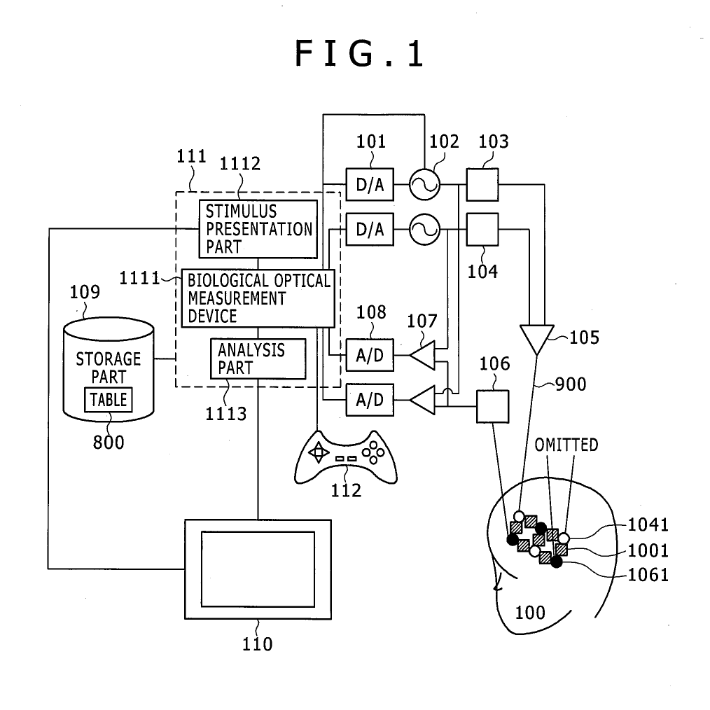

FIG. 1 shows the general configuration of a biological optical measurement device according to the present invention. The biological optical measurement device according to the present embodiment includes: one or a plurality of light irradiation parts 1041 for irradiating an examinee with light; and one or a plurality of light detection parts 1061 for detecting light transmitted or reflected from the examinee. The light irradiation parts 1041 and the light detection parts 1061 include a plurality of measurement points 1001 in a plurality of combinations. Further, the biological optical measurement device according to the present invention includes: a display part 110 for presenting a task to an examinee 100; a storage part 109 for storing various types of information on the task presentation methods and the biological optical measurement results; various tables 800 stored in the storage part to show information such as the task type and the presentation order; and an input part 112 for obtaining an answer from the examinee 100. The biological optical measurement device also includes a calculation part 111 for performing measurement, stimulus presentation, and analysis. The calculation part 111 includes: a stimulus presentation part 1112 for controlling presentation of the tasks in the display part 110; a biological optical measurement part for controlling the light irradiation of the light irradiation parts 1041, converting received optical signals of the light detection parts 1061 into hemoglobin signals, and obtaining answers from the examinee 100 through the input part 112; and an analysis part 1113 for calculating or statistically processing the brain activity values from the hemoglobin signals and storing the brain activity values in the storage part 109, while controlling the biological optical measurement part 1111 by referring to the information on the biological optical measurement results stored in the storage part 109.

Here, the light irradiation parts 1041 emit light of two wavelengths in the range about 600 to 900 nm that can pass through the living body. The specific irradiation method is as follows. The biological optical measurement part 1111 of the calculation part 111 generates light source driving information. A digital/analog converter 101 converts the generated light source driving information into an analog signal. Then, a modulator 102 converts the analog signal into a light source driving signal. Light sources 103 and 104 (laser diode (LD) or light-emitting diode (LED)) emit light of two wavelengths by the source driving signal. A light mixer 105 mixes the light of two wavelengths, which is guided to the light irradiation part 1041 by an optical fiber 900 to irradiate the examinee 100. It is also possible that the LD or LED emitting the light of two wavelengths is integrated into one package, so that the package is directly brought into contact with the examinee 100 to irradiate the examinee 100. The light detection part 1061 guides the light by the optical fiber 900 contacting with the examinee. The light is received by the light detecter 106 (silicon photodiode, avalanche photodiode, photomultiplier, and the like). Then, the received signal is extracted by a lock-in amplifier 107 and an analog/digital converter 108, and transmitted to the biological optical measurement part 1111. Alternatively, the light detecter 106 and the analog/digital converter 108 are integrated into one package, so that the package is brought into contact with the examinee 100 to detect light directly. Then, the software in the biological optical measurement part 1111 calculates the lock-in processing for the digital signal to extract the received light signal.

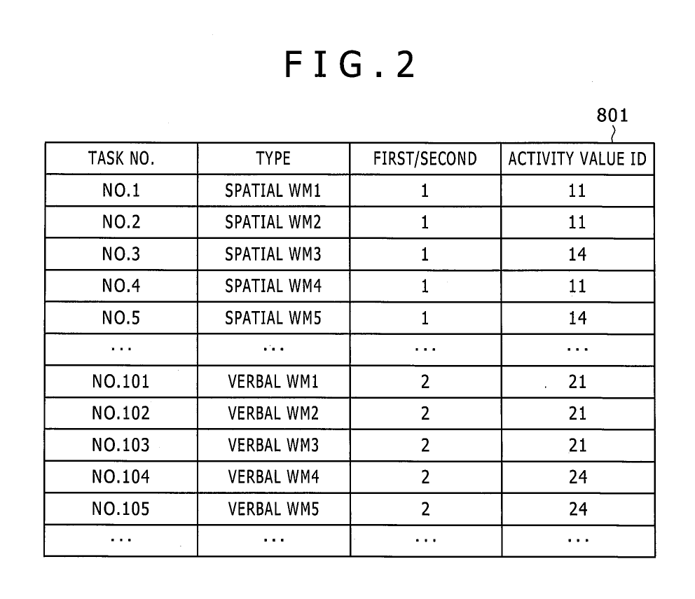

Further, the biological optical measurement device includes: the display part 110 for presenting a plurality of types of tasks (first and second tasks) to the examinee 100; and the calculation part 111 for calculating the brain activity signal in each measurement point 1001 of the examinee 100. The stimulus presentation part 1112 of the calculation part 111 presents a task in the display part 110 based on the table 800 recorded in the storage part 109. Table 801 shown in FIG. 2 is an example of the table 800. The table 801 contains the task number, the task type (here, spatial WM task or verbal WM task), and the information on whether the task is the first task or the second task. Here, as described above in the “supporting research”, the spatial WM task is the first task and the verbal WM task is the second task. Note that the “first” and “second” represent the presentation order of the tasks, which means that the first task is presented a certain number of times and then the second task is presented. The stimulus presentation part 1112 reads the task information to be presented from the table 801 in advance. Then, the stimulus presentation part 1112 presents the task according to the presentation order, for example, in the order A shown in FIG. 7A.

The analysis part 1113 of the calculation part 111 obtains the brain activity signal in each measurement point 1001 of the examinee 100 for the first task, and the brain activity signal in the measurement point 1001 of the examinee 100 for the second task, respectively. Then, the analysis part 1113 calculates the relative value of the respective brain activity signals. First, the analysis part 1113 calculates the brain activity signal in the measurement point 1001 based on the equations 1 and 2 shown in FIG. 10. Here, the values Act_{—}1(1), Act_{—}1(2), . . . , Act_{—}1(n) in the equation 1 represent the brain activity signals of the first block, the second block, and so on, to the n-th block, respectively. For example, as described above in the “supporting research”, this is the average activity value of the oxygenated Hb signals in each block in the activity time from 5 to 8.5 seconds after the presentation of S1. The values Act_{—}2(1), Act_{—}2(2), . . . in the equation 2 are the same as those described above and interpreted by replacing the “first task” with the “second task”. Based on the equations 1 and 2, the analysis part 1113 calculates average values Mean_{—}1 and Mean_{—}2 of the brain activity signals from each of the blocks for the first and second tasks. Then, the analysis part 1113 calculates the relative value of the brain activity values for the first and second tasks. For example, based on the equation 3, the analysis part 1113 obtains mood index D_index from Mean_{—}1 and Mean_{—}2. It is also possible to add a weight to each brain activity signal as shown in the equation 4 in FIG. 10. Here, the values k1 and k2 in the equation 4 are the weight coefficients of Mean_{—}1 and Mean_{—}2, respectively.

Further, the calculation method of the relative value may be the t value for the difference between the brain activity signals for the first and second tasks. The calculation method of the t value is based on the equation 5, in which σ_{—}1 and σ_{—}2 are the standard deviations of the brain activity signals in each of the blocks for the first and second tasks, respectively, and n1 and n2 are the numbers of blocks in the first and second tasks, respectively.

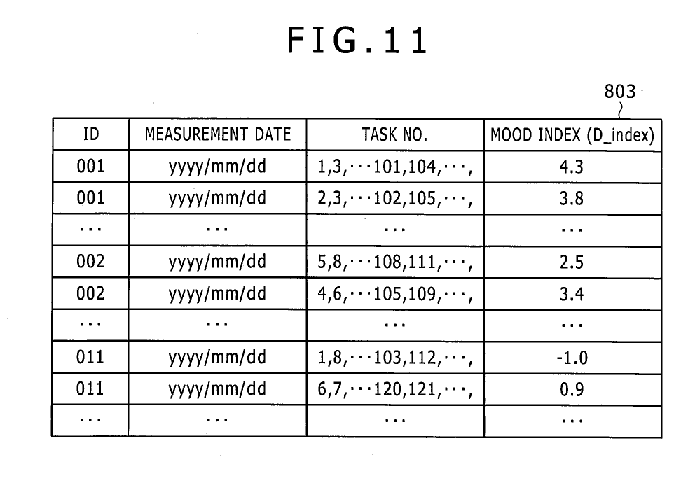

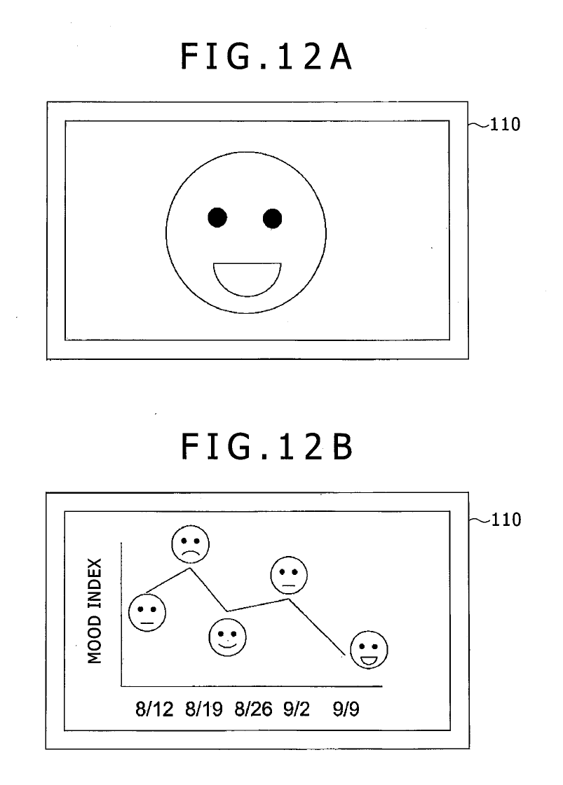

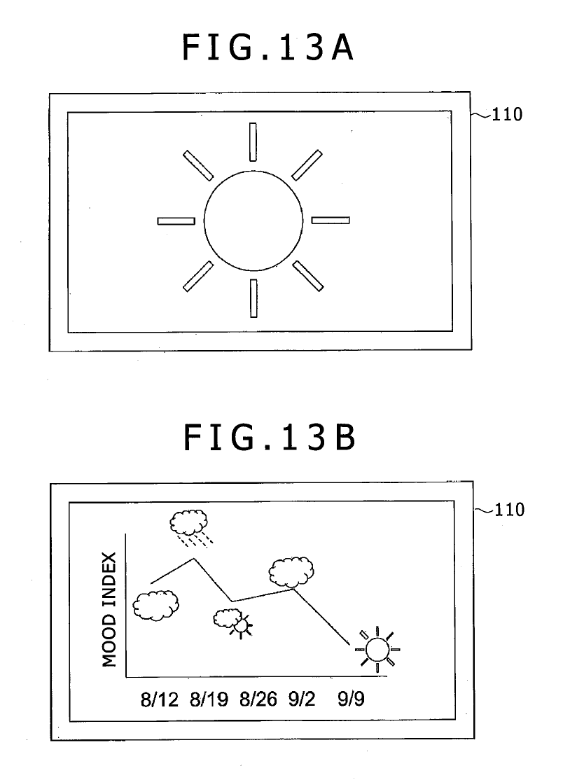

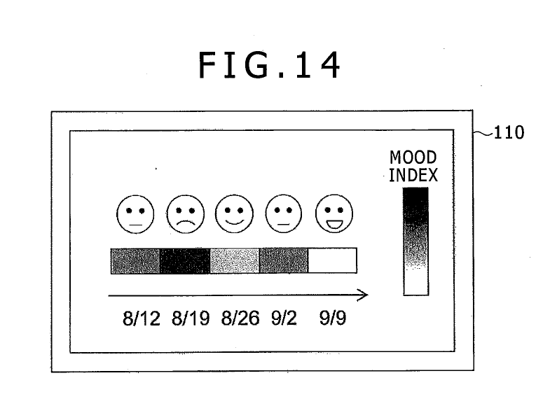

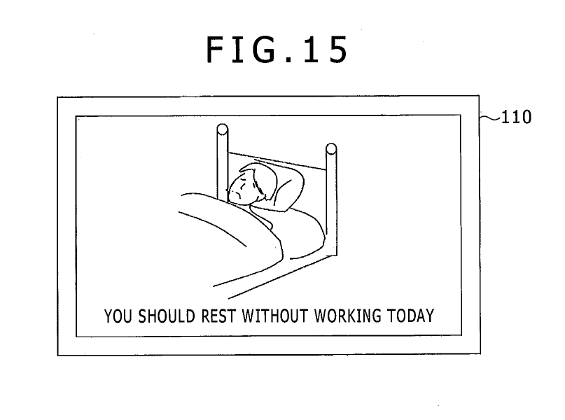

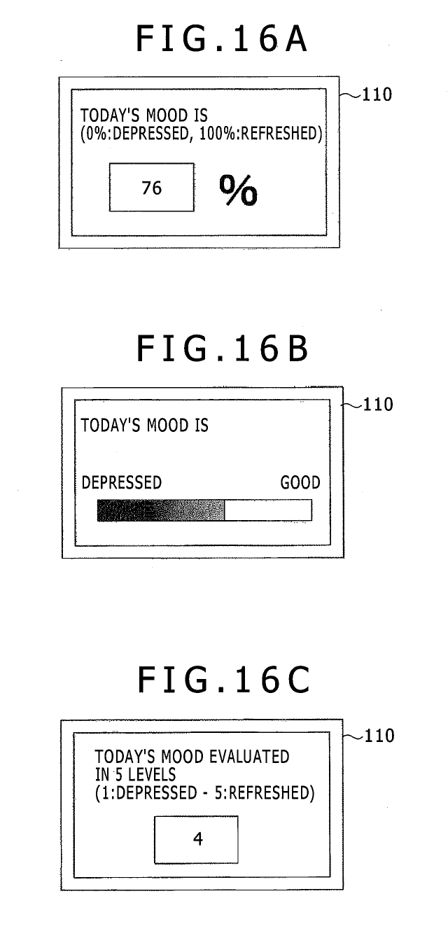

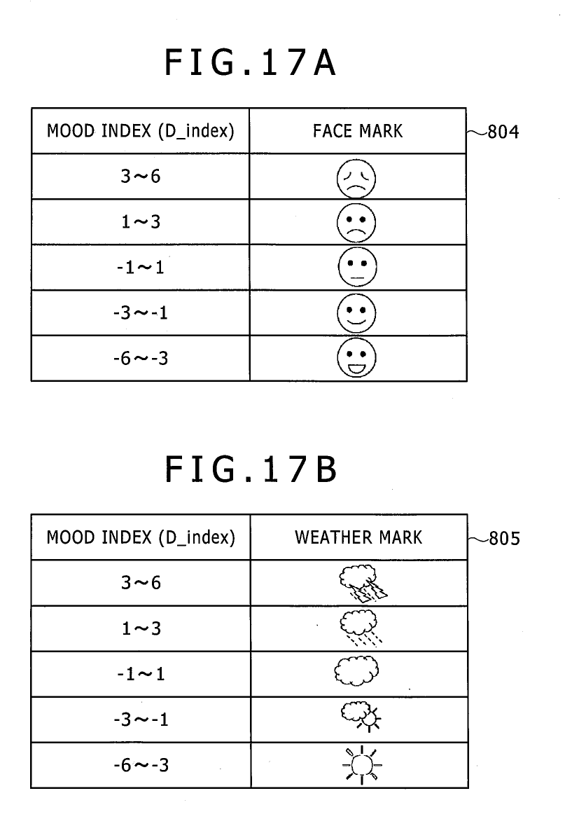

The analysis part 1113 stores the mood index D_index calculated as described above, in the storage part 109. More specifically, the ID for identifying the examinee to be measured, the measurement date, the used task number, and the mood index D_index are associated with each other as a table 803 shown in FIG. 11. Then, the table 803 is stored in the storage part 109. Further, the analysis part 1113 displays the calculated mood index D_index in the display part 110, for example, as shown in FIGS. 12 to 16. FIGS. 12A and 12B are an example of showing the mood index with a face mark. For example, as shown in FIG. 17A, the storage part 109 stores a table 804 in which mood indices and corresponding face marks are associated with each other. The analysis part 1113 calculates the mood index D_index, reads the corresponding face mark from the storage part 109 by referring to the table 804, and displays the particular face mark in the display part 110 as shown in FIG. 12A. Further, the analysis part 1113 can also read the past mood indices of the target examinee by referring to the table 803, select each corresponding face mark by referring to the table 804, and display a graph of the mood indices D_index with the corresponding face marks as shown in FIG. 12B. FIG. 13 is a similar example of the display with the weather mark in place of the face mark. In this case, the analysis part 1113 selects the weather mark corresponding to the calculated mood index D_index by referring to the table 804 shown in FIG. 17B, and displays the results in the display part 110 as shown in FIGS. 13A and 13B. FIG. 14 is an example in which the analysis part 1113 reads the past mood indices D_index from the table 803, associates the mood indices with face marks, displays the results in a color bar graph in the display part 109. FIG. 15 is an example in which the analysis part 1113 displays an image to prompt the examinee to rest in the display part 110, when the calculated mood index D_index is below a certain threshold. FIGS. 16A, 16B, and 16C are an example in which the mood index D_index is displayed in a percentage (FIG. 16A), a bar plot (FIG. 16B), and a five-stage evaluation (FIG. 16C).

With the configuration described above, it is possible to provide an appropriate task presentation order to obtain the index related to the depressed mood by comparing the brain activity signals for each of the different tasks. Further, it is also possible to provide feedback to the examinees by displaying the mood index in the display part 110, which allows the examinees to recognize their objective mood states and the changes in the mood states.

### Second Embodiment

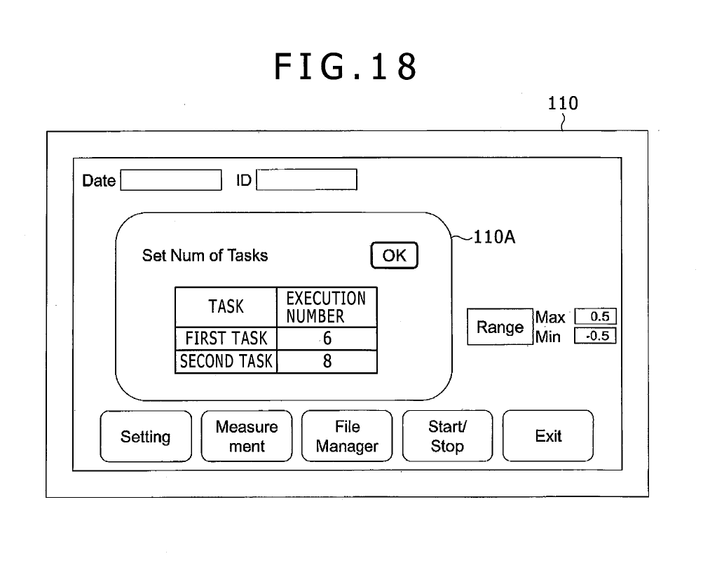

Next, another embodiment of a biological optical measurement device according to the present invention will be described. FIG. 18 is an example of the measurement setting screen, showing a task number set display 110A displayed in the display part 110. The task number set display 110A allows the stimulus presentation part 1112 to input and set the presentation number of the first task and the presentation number of the second task, respectively. The stimulus presentation part 1112 receives the input of the task number set display 110A, and displays the first and second tasks in the display part 110 for the set number, for example, according to the sequence as shown in FIG. 7A. Then, the biological optical measurement part 1111 and the analysis part 1113 perform measurement and analysis according to the number displayed in the display part 110. According to the present embodiment, it is possible to provide the task presentation number in response to the request of the user.

### Third Embodiment

Next, still another embodiment of a biological optical measurement device according to the present invention will be described. In the present embodiment, a configuration for providing an appropriate task presentation number based on the database will be described.

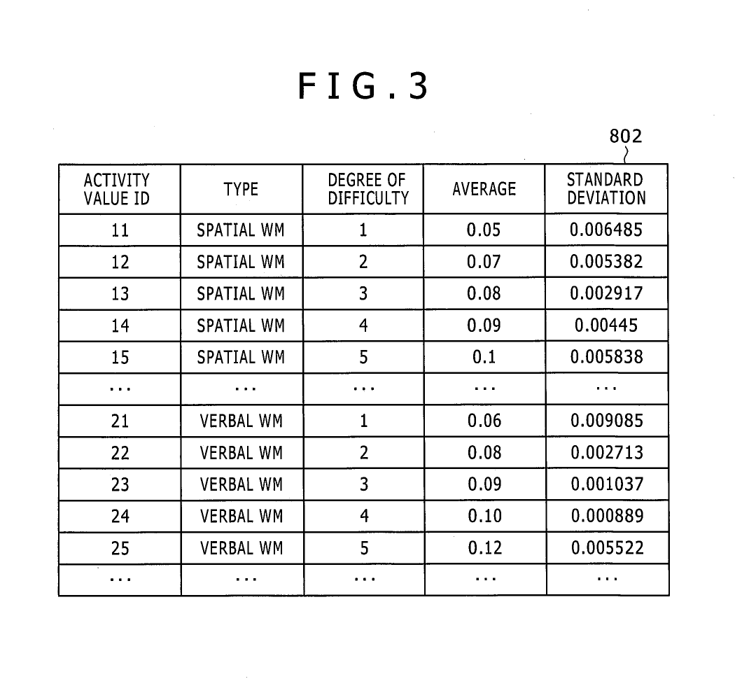

FIG. 3 shows a database 802 showing the degree of difficulty of the spatial and verbal WM tasks and the corresponding brain activity values. The database 802 is stored in the storage part 109, in which the “activity value ID” is given to each task type and the degree of difficulty. Further, the average and standard deviation of the brain activity values obtained from a large number of examinees are recorded for each task type and degree of difficulty identified by the activity value ID. The database 802 is updated to include the newly calculated brain activity value. Further, the activity value ID of the database 802 is associated with each task, and recorded in the table 801 that shows the stimulus presentation order.

Here, as described above in the “supporting research”, the brain activity signal for the spatial WM task has no statistically significant correlation with the POMS depression score, so that it is suggested that the particular brain activity signal is in a certain range not strongly dependent on the mood state. In other words, in the present embodiment, the spatial WM task (first task) allows the examinee to complete the task at the time when the brain activity signal enters a certain range obtained from the database 802 in the range of the predetermined task presentation number, and move to the verbal WM task (second task).

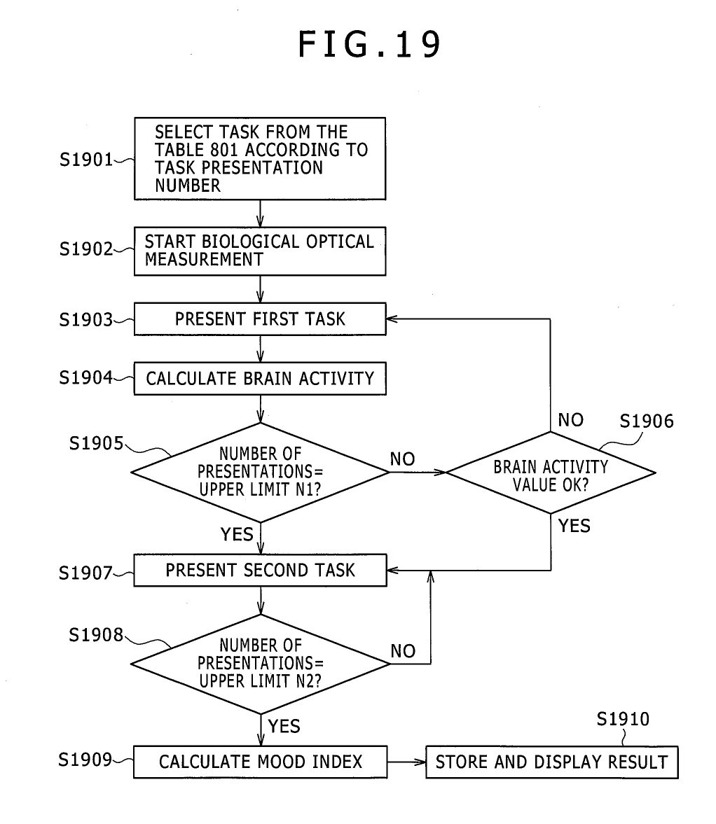

More specifically, this is according to the following procedure. FIG. 19 is a flow chart of the procedure according to the present embodiment. In step S1901, the stimulus presentation part 1112 refers to the predetermined task presentation number or the task presentation number set in the task number set display 110A, to select the task according to the number from the table 801. Here, the presentation number of the first task is N1, and the presentation number of the second task is N2. It is assumed that tasks of the same type are selected from those with the same activity ID in the table 801. In step S1902, the biological optical measurement part 1111 starts the biological optical measurement. In step S1903, the stimulus presentation part 1112 displays the first task in the display part 110. In Step S1904, the analysis part 1113 calculates the brain activity value for the first task, for example, according to the equation 1 shown in FIG. 10. In step S1905, the analysis part 1113 determines whether the presentation number of the first task reaches the set number N1. If YES, the stimulus presentation part 1112 displays a message “move to the second task” in the display part 110. Then, the process proceeds to step S1907. If NO, the process proceeds to step S1906. In step S1906, the analysis part 1113 determines whether the brain activity value is in a certain range specified by the activity value ID. For example, the analysis part 1113 refers to the activity value ID of the database 802 stored in the storage part 109, to determine whether the obtained brain activity value is in the range of the average plus or minus the standard deviation. If YES, the stimulus presentation part 1112 displays a message “move to the second task” in the display part 110. Then, the process proceeds to step S1907. If NO, the process returns to step S1903 to present the first task. In step S1907, the second task is presented, and in step S1908, the second task is presented until the number reaches the set presentation number N2. In step S1908, when the number reaches the presentation number N2, the process proceeds to step S1909 to calculate the mood index D_index, for example, according to the equations 1 to 3 shown in FIG. 10. Then, in step S1910, the stimulus presentation part 1112 stores the mood index D_index in the storage part 109, while displaying the mood index D_index in the display part 110, for example, by the method shown in FIGS. 12 to 16.

According to the present embodiment, by determining whether the brain activity value for the first task (spatial WM task) is in a certain range, it is possible to move to the second task even if the task presentation number does not reach a predetermined number. As a result, the measurement time is reduced and the load on the examinee is reduced.

Note that in the present embodiment, the average and standard deviation described in the database 802 is an example of the statistics for a large number of examinees. It goes without saying that the average and standard deviation can be replaced by the median value, the standard error of the mean, or other various statistical indices.

### Fourth Embodiment

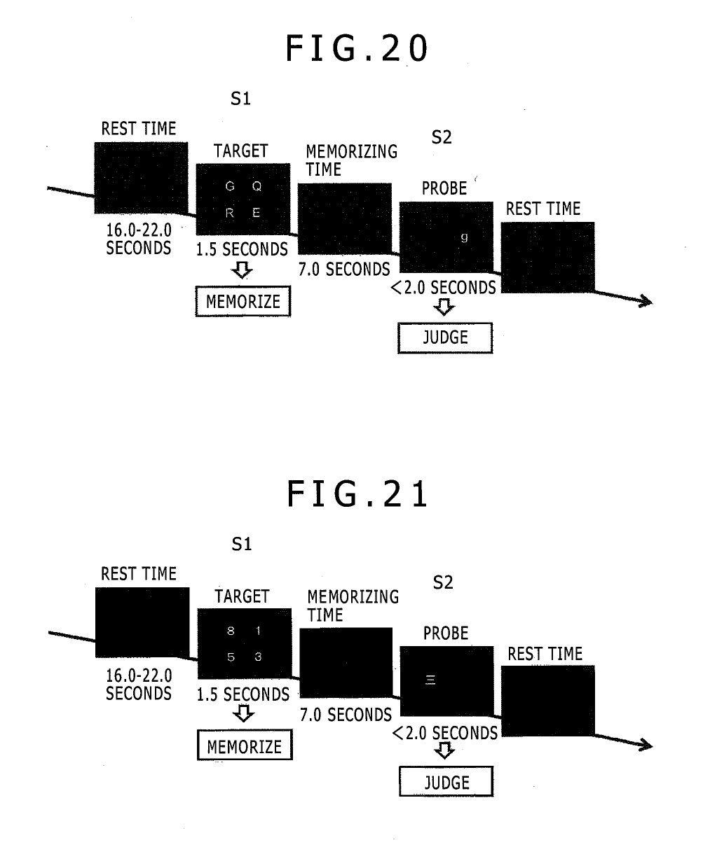

Next, still another embodiment of a biological optical device according to the present invention will be described. FIG. 20 is an example in which the verbal WM task shown in FIG. 6 is replaced by a verbal WM task with alphabet letters. In the present embodiment, the examinee should memorize capital alphabet letters in the first image (S1), and judge whether a small alphabet letter presented in the second image (S2) matches with any of the memorized letters in S1. According to the present embodiment, it is possible to evaluate the mood of the examinees more familiar with the alphabet than Japanese, in the same way as in the embodiments described above. Further, FIG. 21 is an example in which the verbal WM task shown in FIG. 6 is replaced by a verbal WM task with numbers and Chinese characters. The examinee should memorize numbers in the first image (S1), and judge whether one Chinese character presented in the second image (S2) matches with any of the numbers memorized in the first image (S1). According to the present embodiment, it is possible to evaluate the mood of the examinee more familiar with Chinese characters than Japanese, in the same way as in the embodiments described above.

1. A biological optical measurement device comprising:
one or a plurality of light irradiation parts for irradiating an examinee with light;
one or a plurality of light detection parts for detecting light transmitted or reflected from the examinee;
a plurality of measurement points formed by a plurality of combinations of the light irradiation part and the light detection part; and
a stimulus presentation part for presenting a plurality of different types of tasks to the examinee,
wherein the stimulus presentation part presents a first task once or a plurality of times, and then presents a second task a plurality of times,
wherein the calculation part calculates a hemoglobin signal based on the measurement result of a predetermined measurement point for the first task, as well as a hemoglobin signal based on the measurement result of a predetermined measurement point for the second task, and
wherein the calculation part calculates quantitative values using the obtained hemoglobin signals.

2. The biological optical measurement device according to claim 1,
wherein in particular a non-verbal working memory task is used as the first task, and a verbal working memory task is used as the second task.

3. The biological optical measurement device according to claim 1,
wherein in particular the presentation number of the first task and the presentation number of the second task are set.

4. The biological optical measurement device according to claim 1, in particular comprising a database including a-statistical values of a large number of biological optical measurement results for the first task, in the storage part,
wherein the calculation part compares the biological optical measurement result for the first task with the statistical values of the database, to determine whether the first task should be continued or completed.

5. The biological optical measurement device according to claim 1, comprising a storage part for storing the quantitative values.

6. A biological optical measurement device comprising:
one or a plurality of light irradiation parts for irradiating an examinee with light;
one or a plurality of light detection parts for detecting light transmitted or reflected from the examinee;
a plurality of measurement points formed by a plurality of combinations of the light irradiation part and the light detection part;
a stimulus presentation part for presenting a plurality of different types of tasks (first and second tasks) to the examinee;
a calculation part for calculating hemoglobin signals based on the changes in the concentration of oxygenated and deoxygenated hemoglobin in the examinee, from the intensity of the light detected by the light detection part;
a storage part for storing the hemoglobin signals; and
various tables showing the task type, the presentation order, and the like, in the storage part,
wherein the stimulus part presents the first task once or a plurality of times, and then presents the second task a plurality of times,
wherein the calculation part calculates a hemoglobin signal of a predetermined point for the first task, as well as a hemoglobin signal of a predetermined point for the second task, and
wherein the calculation part calculates quantitative values using the obtained hemoglobin signals.

7. A stimulus presentation method for presenting a visual stimulus in a display part, in order to measure the state of the brain by means of a biological optical measurement device,
wherein the stimulus presentation method presents a non-verbal working memory task a plurality of times, and then presents a verbal working memory task a plurality of times.

8. A non-transitory computer-readable medium containing a stimulus presentation program for presenting a visual stimulus in a display part by a calculation part, in order to measure the state of the brain by means of a biological optical measurement device,
wherein the calculation part allows the non-verbal working memory task to be presented a plurality of times at predetermined intervals, and then allows the verbal working memory task to be presented a plurality of times.
